(19) United States
(12) Patent Application Publication
(10) Pub. No.: US 2004/0152210 A1
(43) Pub. Date: Aug. 5, 2004

(54) Method for the relative determination of physicochemical properties

(76) Inventors: Thomas Joos, Tuebingen (DE); Dieter Stoll, Reurlingen (DE); Markus Templin, Tuebingen (DE); Bernhard Virnekaes, Gauting (DE); Ralf Ostendorp, Muenchcn (DE)

Correspondence Address: KNOBBE MARTENS OLSON & BEAR LLP, 2040 MAIN STREET, FOURTEENTH FLOOR, IRVINE, CA, 92614, US

(21) Appl. No.: 10/688,137

(22) Filed: Oct. 17, 2003

Related U.S. Application Data
(63) Continuation of application No. PCT/EP02/04281, filed on Apr. 18, 2002.

(30) Foreign Application Priority Data
Apr. 19, 2001 (EP) 01 109 705.2

(51) Int. Cl. G01N 33/543

(52) U.S. Cl. 436/518

(57) ABSTRACT

A method for determining a first physicochemical property of at least two compounds relative to each other is described which uses a second physicochemical property, whereby determination of the first property depends on a third, undetermined physical property, and the first property depends on the composition of the respective compound. Determination of the second property depends on the third property as well, but the second property does not depend on the composition of the respective compound. For each compound a first value for the first property is measured under certain, preferably equilibrium conditions, and simultaneously a second value for the second physical property under said certain conditions is measured. The first property for each compound relative to the other compound is determined by using the first and second values.

### CROSS-REFERENCES TO RELATED APPLICATIONS

This application is a continuation of copending International Patent Application PCT/EP02/04281 filed on Apr. 18, 2002, and designating the US, which was published under PCT Article 21(2) in English, and claims priority of European Patent Application EP 01 109 705.2, filed on Apr. 19, 2001, which is incorporated herein by reference.

### BACKGROUND OF THE INVENTION

1. Field of the Invention

The present invention relates to a method for determining a first physicochemical property of at least two compounds relative to each other using a second physicochemical property, wherein said first property depends (i) on a third, undetermined physicochemical property, and (ii) on the composition of the respective compound, and wherein said second property depends (i) on said third property as well, but (ii) does not depend on the composition of the respective compound. Particularly, the method relates to the relative determination of binding affinities in a parallelized way.

2. Related Prior Art

Since mid of the 1980s, enormous progress has been made in the fields of Combinatorial Chemistry and Combinatorial Biology. Countless libraries of non-proteinaceous, peptide and protein libraries have been designed and synthesized. In parallel, highly sophisticated screening and selection technologies have been developed, in all cases incorporating means for the identification of individual compounds having a desired property.

Most often, the hits obtained after screening and/or selection are not yet the final products, and the individual compounds obtained therein have to be characterized in order to identify the best choice for further detailed analysis and fine-tuning of specific features. This step may be decisive for the success of further optimization strategies, and thus, for the over-all success of costly product development.

For example, one feature, which often is of central importance, is the binding affinity of individual compounds obtained by screening a library for binding to a given target. In most cases, a certain threshold affinity has to be reached, or a certain range of affinities has to be met by a positive hit in order to take this hit into further rounds of optimization. However, in known methods for determining the affinity of a compound one needs to know the concentration of the compound. But in most cases the various compounds of a chemical library or members of a peptide or protein library cannot be synthesized or expressed in a standardized way to yield identical amounts of material. Thus, every compound or member has to be synthesized or expressed, and the concentration be determined individually. This requires an enormous amount of work.

### SUMMARY OF THE INVENTION

Thus, the technical problem underlying the present invention is to develop a simple, reliable system which enables the rapid determination of a physicochemical property of different molecules relative to each other without being able to directly determine all parameters on which that property depends.

The solution to this technical problem is achieved by providing the embodiments characterized in the claims. Accordingly, the present invention allows to easily determine properties such as the binding affinity of two or more candidate binders to a target without knowing the concentration of the individual candidates. The technical approach of the present invention, i.e. the simultaneous determination of a second property which is independent of the composition of the individual candidate, is neither provided nor suggested by the prior art.

Accordingly, the present invention according to one object relates to a method for determining a first physicochemical property of at least two compounds relative to each other by using a second physicochemical property, wherein determination of said first property depends on a third, undetermined physicochemical property, and said first property depends on the composition of the respective compound, and wherein determination of said second property depends on said third property as well, but said second property does not depend on the composition of the respective compound, for each compound comprising the steps of:

a) measuring a first value for said first property under certain, preferably equilibrium conditions; and

b) simultaneously measuring a second value for said second physicochemical property under said certain conditions;

and determining said first property for each compound relative to the other compound(s) by using said first and second values.

In the context of the present invention, the expression “certain conditions” relates to experimentally reproducible conditions or states of e.g. an assay. Thus, certain conditions can e.g. mean a certain point in time of an assay, a certain turnover of an enzymatic reaction or, preferably, equilibrium.

Further, in the context of the present invention, the term physicochemical property relates to properties which can be determined by physicochemical measurements, and include, without being limited to, properties such as concentration, affinity constant of binding to a target, activity of a DNA promoter region, enzymatic activity.

For example, the first property may be the promoter activities of a series of different promoter sequences comprised in a collection of DNA vector molecules. The direct measurement of protein being expressed from DNA under control of the individual promoter region would not be possible since the expression yield would depend on a third, undetermined property, which is the individual expression condition of individual cells harboring the DNA vector molecules. By measuring the expression yield of a second protein under the control of a second, fixed promoter region comprised in each of said collection of DNA vector molecules, a relative determination of the first property can be obtained.

In a preferred embodiment of the present invention, the third property is the concentration of each of the compounds.

In a further preferred embodiment, the first property is the affinity constant of binding to a first target.

In principle, the affinity of compound and target in the meaning of this invention can relate to any kind of ligand binding assay, like but not limited to e.g. interaction of a first and a second protein, antigen and antibody, receptor and ligand, enzyme and substrate, DNA and protein, RNA and protein.

According to another object of the method of the present invention, said first target is an antigen, and said at least two compounds comprise the variable domains of different antibodies binding to said antigen.

In this context, “antibody” is used as a synonym for “immunoglobulin”. Immunoglobulin fragments comprising the variable domains of antibodies according to the present invention may be Fv (Skerra & Plueckthun, 1988), scFv (Bird et al., 1988; Huston et al., 1988), disulfide-linked Fv (Glockshuber et al., 1992; Brinkmann et al., 1993), Fab, (Fab′)2 fragments or other fragments well-known to the practitioner skilled in the art. Particularly preferred is the scFv fragment format. Further preferred is the Fab fragment format.

Particularly preferred is a method, wherein said second property is the affinity constant of binding to a second target.

Further preferred is a method, wherein said second target is an antibody or functional fragment thereof with specificity for an antibody-binding site comprised in each of said at least two compounds.

In the case, where the compounds are antibodies or functional fragments thereof, said second target can be an antibody with binding specificity for a constant part of each of the antibodies or functional fragments thereof, e.g. for a peptidic tag linked to an scFv fragment, or for an epitope in one or both constant domains in an Fab fragment.

Further, said compounds may be antigens of interest and said first target may be a first antibody, so that different antigens may be screened and ranked according to their binding to said first antibody using the binding of the antigens to a second target that can be a second antibody with constant affinity to all antigens analyzed.

In view of the above, yet another object of the invention is a method for ranking at least two compounds relative to each other with respect to their first affinity constant of binding to a first target, by using their second affinity constant of binding to a second target, wherein determination of said first affinity constant depends on the concentration of each of said compounds, and said first affinity constant depends on the composition of the respective compound, and wherein determination of said second affinity constant depends on said concentration as well, but said second affinity constant does not depend on the composition of the respective compound, for each compound comprising the steps of:

a) measuring a first value for said first affinity constant under equilibrium conditions; and

b) simultaneously measuring a second value for said second affinity constant under said equilibrium conditions;

and determining said first affinity constant for each compound relative to the other compound(s) by using said first and second values.

Yet further preferred is a method, wherein said steps (a) and (b) are performed in parallel for multiple compounds, and wherein each compound is contained in one well or an otherwise defined area of a substrate.

Said substrate can e.g. be a microtiter plate or a glass slide having thereon defined distinct areas by e.g. hydrophobic ridges, protrusions or stripes.

Particularly preferred is a method, wherein said steps (a) and (b) are performed in parallel for multiple compounds, and wherein each compound is contained in one spot of a microarray.

The present invention further relates to a method, wherein each of said at least two compounds is in solution.

Another object is a method, wherein said steps (a) and (b) are being performed by simultaneously contacting said solution with said first and said second target, each being immobilized on a solid phase, and wherein the amounts of compound binding to said first and second target are measured for each compound.

According to a further object of the present invention, said first and said second target are being immobilized to different subsets of microspheres.

Preferred is also a method, wherein said different subsets are characterized by different fluorescence labels.

Further, the method comprises the step of identifying binding of a compound to said first or second subset of microspheres by binding of a fluorescence label to the compound.

In the case, where the compounds are antibodies or functional fragments thereof, the binding of a fluorescence label to the antibodies or functional fragments thereof being achieved by a fluorescence-labeled detection antibody with binding specificity for a constant part of each of the antibodies or functional fragments thereof, e.g. a peptidic tag linked to an scFv fragment, or one or both constant domains in an Fab fragment, wherein binding of said detection antibody is independent from binding of said second target.

In a yet further preferred embodiment of the present invention, each of said at least two compounds is immobilized to the surface of a solid phase.

In a particularly preferred embodiment, the invention relates to a method, wherein said steps (a) and (b) are being performed by simultaneously contacting said immobilized compound with known amounts of said first and said second target in solution, and wherein the relative amounts of first and second target binding to said immobilized compound are measured.

The solid phase can either be a planar microarray like e.g. glass slides with activated surface, or bead-based systems with microspheres. The microspheres can be divided into subsets, each subset being characterized e.g. by a specific color, so that like with planar microarrays with a set of microspheres comprising different subsets, a number of parameters can be determined in the scope of the present application.

In this connection the invention according to another object relates to a set of at least two different subsets of microspheres for performing the method according to the invention, the microspheres in each subset thereof having immobilized thereon a target for compounds to be ranked with respect to their first physicochemical property, the targets in different subsets being different.

Preferably the kit contains a set of at least two compounds, each being able to bind to a respective one of the at least two targets, i.e. each a compound for a target.

Optionally, further subsets of microspheres can be used as well. Thus, the different subsets of microspheres can be used for performing the method according to the present invention, wherein the microspheres in each subset thereof having immobilized thereon a target for compounds to be ranked with respect to their binding to the first target, the targets in different subsets being different.

According to another object, a kit for ranking antibodies with respect to their affinity to a target comprises a set of at least two different subsets of microspheres, the microspheres in a first subset having immobilized thereon a capture molecule for antibodies, the microspheres in a second subset thereof being pre-activated, so that the target can be immobilized thereon. Preferably, the kit also comprises a detection antibody.

Thus, this kit provides in the first subset of microspheres a capture molecule, e.g. an anti-IgG, that is used for measuring the concentration of the antibody. The second subset of microspheres is prepared to have immobilized thereon a target, i.e. an antigen, the antibodies being ranked with respect to their affinity to this antigen. The optionally also provided detection antibody binds to antibodies bound either to the capture molecule or to the antigen. With the first subset of microspheres the detection antibody gives a first signal, and with the second subset of microspheres a second signal, by dividing the first signal by the second signal a ranking value Q is determined for each antibody tested with this kit.

Of course, it is also possible to immobilize compounds on the beads and to have the targets in solution, instead of having the targets immobilized on different subsets of microspheres and having the compounds in different solutions.

Using beads as solid phase has the further advantage that the measurements can be made on equilibrium conditions, since no washing steps are required. All that is necessary is to mix the solution containing the compound with the subsets of beads having immobilized thereon the different targets, and then to measure the fluorescence signals of the beads and of the complexes formed by beads and the compound bound to the target.

It is possible to determine the affinity of the compound contained in the solution to more than two different binding molecules. Thereby, cross reactivity can not only be detected but also quantified, what is a very important point for characterizing antibodies. In this connection, e.g. 3, 4 or 5 subsets of beads or a planar array having immobilized 3, 4 or 5 different binding molecules in one area can be used.

Further, it is possible to split an antigen into different small epitopes and to immobilize the different epitopes, e.g. on different subsets of microspheres, and then to apply the inventive method.

A further advantage lies in the fact that only small amounts of immobilized targets are necessary so that measurements are possible under the so-called ambient analyte conditions, i.e. where the forming of the antigen-antibody complex does substantially not change the concentration of the compound in the solution.

The present invention in connection with a further object relates to a kit comprising a first carrier comprising at least two areas for retaining sample solutions; and a second carrier comprising, for each of said areas comprised in said first carrier, at least two positions suitable for the immobilization of at least a first and a second compound, wherein said second carrier and said first carrier can be brought in contact in a way which allows to simultaneously contact each of said solutions with at least said first and second compounds immobilized to said at least two positions, and wherein the amounts of material out of said sample solution binding to said first and second compounds can be measured for each said sample solution.

According to another object, the invention relates to a kit comprising a first carrier comprising a least two areas for retaining sample solutions and a second carrier comprising, for each of said areas comprised in said first carrier, at least two positions suitable for the immobilization of at least a first and a second target, wherein said second carrier and said first carrier can be brought in contact in a way which allows to simultaneously contact each of said solutions with at least said first and second target immobilized to said at least two positions, and wherein the amounts of material out of said sample solution binding to said first and second targets can be measured for each said sample solution.

As outlined above, the areas can be wells of a microtiter plate or defined areas of a substrate, like areas on a glass slide defined by hydrophob surrounding.

### BRIEF DESCRIPTION OF THE FIGURES

FIG. 1 shows equations derived from the mass action law for calculating the relative affinity constant of the binding of an antibody with unknown concentration to an antigen.

FIG. 2 shows a peg cover and microtiter plate for performing the invention.

FIG. 3 shows a table summarizing the results of experiments for ranking the binding of 12 different antibodies to three different antigens.

FIG. 4 shows a plot of relative affinity constant versus affinity value for the binding of 55 antibody fragments to an antigen, the relative affinity constant being obtained with the new method.

### DESCRIPTION OF DETAILED EMBODIMENTS

The following examples illustrate the invention.

### EXAMPLE 1

### Microarray Assay Development for the Determination of the Affinity of Antibody-Antigen Interaction

The determination of the kinetic constants of an antibody is generally performed with kinetic measurement. An affinity constant can also be determined with the mass action law, if the concentrations of antibody [Ab], antigen [Ag] and the antigen-antibody product [Ag-Ab] are known. The determination of these concentrations can be simultaneously performed with microarray technology. Antigens (Ag) and an antibody specific capture molecule (α-Ab) have to be immobilized on such a microarray. An antigen-antibody-product (Ag-Ab) and antiantibody-antibody product(α-Ab-Ab) is observed when the array is incubated with the antibody of interest.

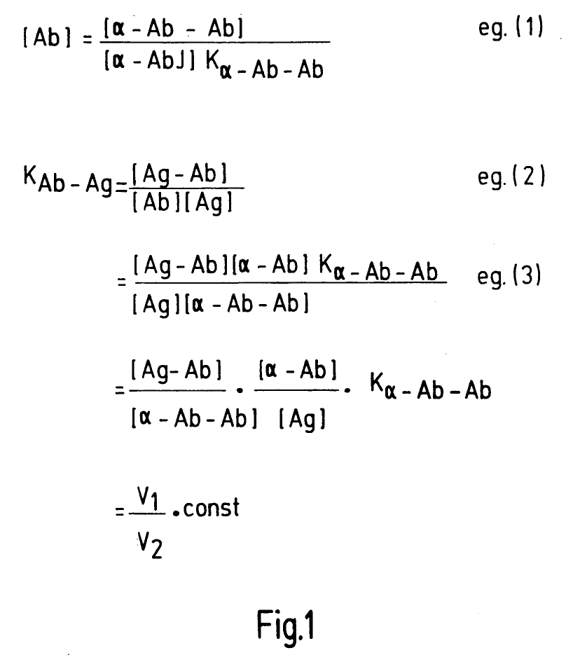

The reactions observed are shown in FIG. 1 as eq. (1) and (2). It can be seen that within equilibrium the desired affinity constant K_{Ag-Ab} can be calculated from eq. (3) and does not depend from the concentration of the antibody to be evaluated. In order to perform a ranking of different antibodies with respect to their binding to the same antigen it is even not necessary to know the affinity constant K_{α-Ab-Ab} since K_{Ag-Ab} can be calculated in relation to K_{α-Ab-Ab}. Even further, the exact amount of Ag and α-Ab has not to be known as long as the values for [Ag] and [α-Ab] are kept constant for the measurements performed for the different antibodies to be evaluated, i.e. as long as the influence of the formation of complexes [Ag-Ab] and [α-Ab-Ag] on the concentrations of [Ag] and [α-Ab] are neglectable. In eq. (3) these three variables—[Ag], [α-Ab] and K_{α-Ab-Ag}—have been summarized in a factor called const.

Thus, from the measured values V(Ag-Ab) and V(α-Ab-Ab) for [Ag-Ab] and [α-Ab-Ab], respectively, a relative affinity constant

Kᵣₑₗ=K_{Ag-Ab}/const=V(Ag-Ab)/V(α-Ab-Ab)

can be calculated so that a relative ranking of the antibodies with respect to their antigen binding can be found.

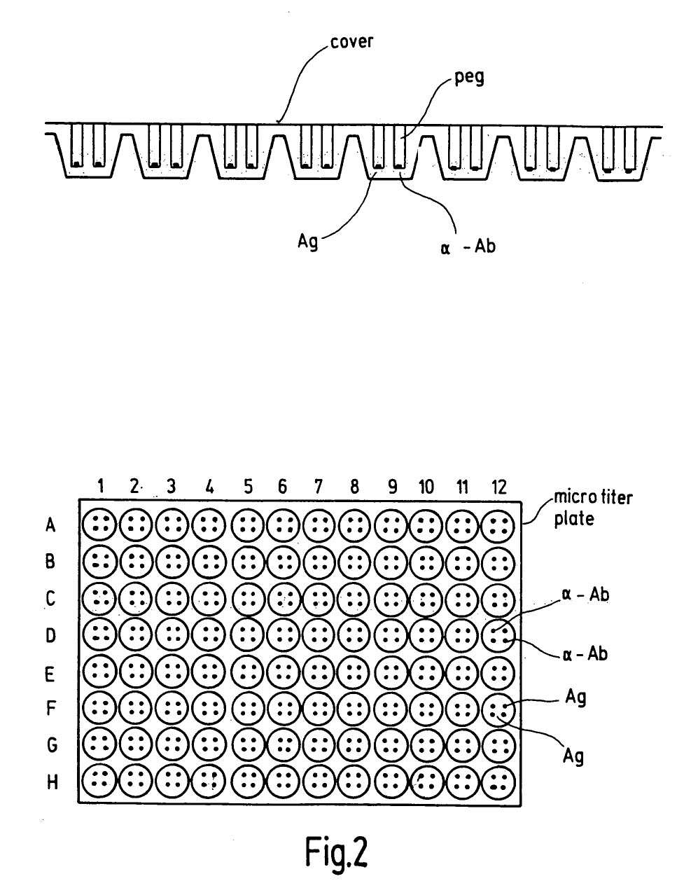

FIG. 2 shows a peg cover and microtiter plate suited for performing the invention. The cover carries in the example shown 384 pegs arranged in 96 groups of each 4 pegs. The microtiter plate has 96 wells, each well receiving 4 pegs. On each 2 pegs out of a group of 4 pegs the antigen and the α-antibody are being loaded using standard methods known in the art. For example, the cover can be laid on a 384 well plate such that each peg protrudes into a distinct well containing either antigen or α-antibody solution. According to another method, the antigen- and α-antibody-molecules can be spotted onto the pegs.

Material and Methods

Material

A protein antigen Ag (MorphoSys, Martinsried);

4 different purified Fab antibody fragments specific for Ag, each containing a FLAG epitope (MorphoSys, Martinsried);

capture antibody: a-hu-Fab (Jackson Laboratories, PA);

Cy5-conjugated a-hu-Fab Fab (Jackson Laboratories, PA) was used as secondary antibody.

Note: when performing the assays the antigen and the antibodies were “unknown compounds” to the inventors, so that the experiments were “blind” experiments in view of proof of principle of the invention.

Corning CMT-GAPS coated slides (gamma-aminopropyl silane activated glass surface; Corning Lifescience, USA) and Telechem CSS slides (aldehyde activated glass surface; Telechem Corp., USA) were used as support material.

Microarrays were created with a GMS 417 microarrayer (MWG,=20 Ebersberg).

Signals were detected using a GMS 418 array scanner (MWG, Ebersberg) and signal processing was performed using the ImaGene 4.0 software.

Blocking buffer: 1.5% BSA, 5% low fat milk powder in PBS

Dilution buffer: 1.5% BSA, 2.5% low fat milk powder, 0.1% Tween-20 in PBS

Methods

The antigen Ag was diluted in a buffer suitable for use with a GMS 417 micro-arrayer. The used buffer (printing buffer) contains 5=μg/ml BSA, 0.02% SDS, 10% glycerol, 0.1 mg/ml Bromphenol Blue in PBS, pH 7.4.=20

An adopted micro-ELISA protocol was used to detect the immobilized proteins. First, the array (approximately 0.5 cm²) was blocked with 80 μl of blocking buffer for 1 h at room temperature. The provided antibodies were diluted in dilution buffer (1/40 for the purified samples) and incubated for 1 h on the array. After washing, the secondary antibody (Cy5-conjugated a-hu-Fab diluted 1/100) was applied for 25 min and after a second washing step the slides were dried.

Results

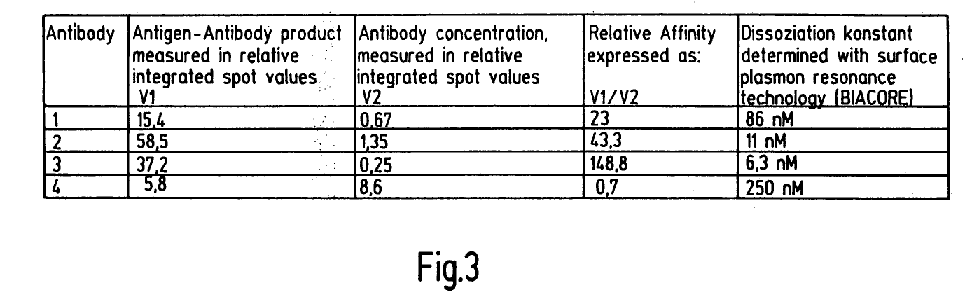

The cy5-signals v1 and v2 for the Ag-Ab and the α-Ab-Ab complex, respectively, were taken for each tested combination of antibody and antigen. FIG. 3 contains a table showing the values for v1 and v2 as well as the quotient Q=v1/v2 that is a direct measure for the relative affinity and, therefore, gives directly the ranking of the different affinities. The ranking found corresponds to the dissociation constant determined with surface plasmon resonance technology (BIACORE).

In order to evaluate the concentration dependency of the new method, for a certain antibody and a certain antigen Q has been determined for a relative antibody concentration. With respect to the concentration of antigen and a-hu-Fab the method works at least over 2 orders of magnitude of relative antibody concentration (data not shown).

### EXAMPLE 2

### Affinity Ranking of Antibody Fragments Using Microspheres

Material

A protein antigen AgB (MorphoSys, Martinsried);

55 different cellular extracts containing Fab antibody fragments specific for AgB, the cellular extracts being at unknown concentrations; the extracts were produced from bacterial clones that express characterized antibody fragments; the affinity value of the antibodies to AgB was determined by surface plasmon resonance measurements (Biacore);

Capture antibody: goat anti human-Fab specific antibody (Jackson Laboratories, PA);

PE-conjugated goat anti human-Fab specific Fab was used as detection antibody;

Luminex 100 System (Luminex Corp., TX);

xMAP Multi-Analyte COOH Microspheres (Luminex Corp., TX, USA);

Activation Buffer: 0.1 M NaH₂PO₄, pH 6.2;

Coupling Buffer: PBS, pH 7.4;

Wash Buffer: PBS, 0.05% TWEEN, pH 7.4;

Blocking/Storage Buffer: PBS, 1% BSA, 0.05% Azide, pH 7.4;

EDC (1-ethyl-3-[3dimethylaminopropyl] carbodiimide hydrochloride), Pierce;

Sulfo-NHS (N-hydroxysulfo-succinimide), Pierce;

all other chemicals were purchased from Sigma Chemical Co. (MO, USA).

Method

The antigen AgB and the goat anti human-Fab specific antibody were coupled to different types of Multi-Analyte COOH Microspheres using the carbodiimide activation chemistry. The coupling reaction was performed as described by the manufacturer using a protein concentration of 30 μg/ml of AgB and 50 μg/ml of anti human-Fab specific antibody.

The affinity ranking experiment was done using 30 μl of a 1:128 dilution of each of the 55 different crude cell extracts; 25 of the samples were measured in duplicate. To the samples approximately 1000 beads of each of the two types, i.e. with target antigen and capture antibody, were added to each sample in a volume of 30 μl and the mixture was incubated for 1 h at room temperature.

30 μl of the labeled detection antibody in a concentration of 10 μg/ml was added and the mixture was incubated for further 45 min.

Results

Signal detection was performed using a Luminex 100 system. 250 beads of each type were counted for determination of the fluorescence signals. The median was determined and used for the calculation of the relative affinity constant. Comparable to Example 1, this constant was obtained by dividing the signal intensity for the antigen bead by the signal intensity for the anti-antibody bead.

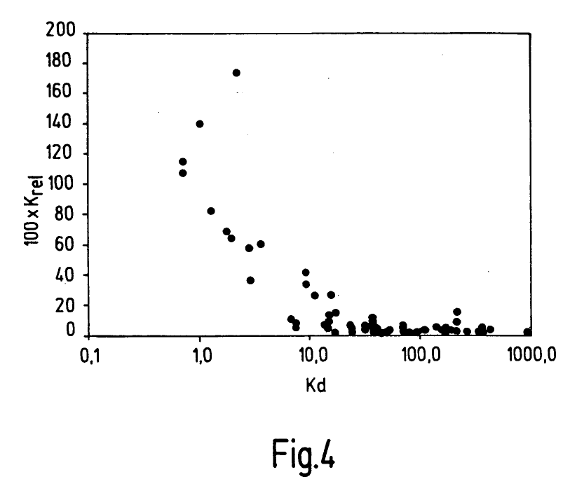

The obtained data are shown in FIG. 4, where the known affinity value determined by surface plasmon resonance measurements K_{D} plotted versus the calculated relative affinity constant Kᵣₑₗ.

It can be seen that antibodies with high affinity to the antigen, i.e. having a K_{D} of less than 20, can be identified and ranked.

What is claimed is:

1. A method for determining a first physicochemical property of at least two compounds relative to each other by using a second physicochemical property, wherein determination of said first property depends on a third, undetermined physicochemical property, and said first property depends on the composition of the respective compound, and wherein determination of said second property depends on said third property as well, but said second property does not depend on the composition of the respective compound, for each compound comprising the steps of:
a) measuring a first value for said first property under certain conditions; and
b) simultaneously measuring a second value for said second physicochemical property under said certain conditions; and
c) determining said first property for each compound relative to the other compound(s) by using said first and second values.

2. The method of claim 1, wherein said third property is the concentration of each of said compounds.

3. The method of claim 2, wherein said first property is the affinity constant of binding to a first target.

4. The method of claim 3, wherein said first target is an antigen, and wherein said at least two compounds comprise the variable domains of different antibodies binding to said antigen.

5. The method of claim 3, wherein said second property is the affinity constant of binding to a second target.

6. The method of claim 5, wherein said second target is an antibody or functional fragment thereof with specificity for an antibody-binding site comprised in each of said at least two compounds.

7. The method according to claim 3, wherein said steps (a) and (b) are performed in parallel for multiple compounds, and wherein each compound is contained in one well or an other-wise defined area of a substrate.

8. The method according to claim 3, wherein said steps (a) and (b) are performed in parallel for multiple compounds, and wherein each compound is contained in one spot of a microarray.

9. The method of claim 1, wherein said second property is the affinity constant of binding to a second target.

10. The method of claim 9, wherein said second target is an antibody or functional fragment thereof with specificity for an antibody-binding site comprised in each of said at least two compounds.

11. The method according to claim 10, wherein said steps (a) and (b) are performed in parallel for multiple compounds, and wherein each compound is contained in one well or an other-wise defined area of a substrate.

12. The method according to claim 10, wherein said steps (a) and (b) are performed in parallel for multiple compounds, and wherein each compound is contained in one spot of a microarray.

13. The method according to claim 1, wherein said steps (a) and (b) are performed in parallel for multiple compounds, and wherein each compound is contained in one well or an other-wise defined area of a substrate.

14. The method according to claim 1, wherein said steps (a) and (b) are performed in parallel for multiple compounds, and wherein each compound is contained in one spot of a microarray.

15. The method of claim 1, wherein each of said at least two compounds is in solution.

16. The method of claim 15, wherein said steps (a) and (b) are being performed by simultaneously contacting said solution with said first and said second target, each target being immobilized on a solid phase, and wherein the amounts of compound binding to said first and second target are measured for each compound.

17. The method of claim 16, wherein said first and said second target are being immobilized to different subsets of microspheres.

18. The method of claim 17, wherein said different subsets are characterized by different fluorescence labels.

19. The method of claim 18, further comprising the step of identifying binding of a compound to said first or second subset of microspheres by binding of a fluorescence label to the compound.

20. The method of claim 1, wherein each of said at least two compounds is immobilized to the surface of a solid phase.

21. The method of claim 20, wherein said steps (a) and (b) are being performed by simultaneously contacting said immobilized compound with known amounts of said first and said second target in solution, and wherein the relative amounts of first and second target binding to said immobilized compound are measured.

22. The method of claim 1, wherein said certain conditions are equilibrium.

23. A kit, comprising
a) a first carrier comprising a least two areas for retaining sample solutions;
b) a second carrier comprising, for each of said areas comprised in said first carrier, at least two positions suitable for the immobilization of at least a first and a second compound, wherein said second carrier and said first carrier can be brought in contact in a way which allows to simultaneously contact each of said solutions with at least said first and second compounds immobilized to said at least two positions, and wherein the amounts of material out of said sample solution binding to said first and second compounds can be measured for each said sample solution.

24. A kit, comprising
a) a first carrier comprising a least two areas for retaining sample solutions;
b) a second carrier comprising, for each of said areas comprised in said first carrier, at least two positions suitable for the immobilization of at least a first and a second target, wherein said second carrier and said first carrier can be brought in contact in a way which allows to simultaneously contact each of said solutions with at least said first and second target immobilized to said at least two positions, and wherein the amounts of material out of said sample solution binding to said first and second targets can be measured for each said sample solution.

25. A kit comprising a set of at least two different subsets of microspheres for performing the method of claim 1, the microspheres in each of said subsets thereof having immobilized thereon a target for compounds to be ranked with respect to the first physicochemical property, the target s in different subsets being different, and preferably a set of at least two compounds, each being able to bind to a respective one of the at least two targets.

26. A kit for ranking antibodies with respect to their affinity to a target, comprising a set of at least two different subsets of microspheres, the microspheres in a first subset having immobilized thereon a capture molecule for antibodies, the microspheres in a second subset being pre-activated so that the target can be immobilized thereon.

27. The kit of claim 26 further comprising a detection antibody.

28. A method for ranking at least two compounds relative to each other with respect to their first affinity constant of binding to a first target, by using their second affinity constant of binding to a second target,
wherein determination of said first affinity constant depends on the concentration of each of said compounds, and said first affinity constant depends on the composition of the respective compound, and
wherein determination of said second affinity constant depends on said concentration as well, but said second affinity constant does not depend on the composition of the respective compound, for each compound comprising the steps of:
a) measuring a first value for said first affinity constant under equilibrium conditions; and
b) simultaneously measuring a second value for said second affinity constant under said equilibrium conditions;
c) and determining said first affinity constant for each compound relative to the other compound(s) by using said first and second values.

29. The method of claim 28, wherein said first target is an antigen, and wherein said at least two compounds comprise the variable domains of different antibodies binding to said antigen.

30. The method of claim 28, wherein said second target is an antibody or functional fragment thereof with specificity for an antibody-binding site comprised in each of said at least two compounds.

31. The method of claim 28, wherein said steps (a) and (b) are performed in parallel for multiple compounds, and wherein each compound is contained in one well or an otherwise defined area of a substrate.

32. The method of claim 28, wherein said steps (a) and (b) are performed in parallel for multiple compounds, and wherein each compound is contained in one spot of a microarray.

33. The method of claim 28, wherein each of said at least two compounds is in solution.

34. The method of claim 33, wherein said steps (a) and (b) are being performed by simultaneously contacting said solution with said first and said second target, each target being immobilized on a solid phase, and wherein the amounts of compound binding to said first and second target are measured for each compound.

35. The method of claim 34, wherein said first and said second target are being immobilized to different subsets of microspheres.

36. The method of claim 35, wherein said different subsets are characterized by different fluorescence labels.

37. The method of claim 36, further comprising the step of identifying binding of a compound to said first or second subset of microspheres by binding of a fluorescence label to the compound.

38. The method of claim 28, wherein each of said at least two compounds is immobilized to the surface of a solid phase.

39. The method of claim 38, wherein said steps (a) and (b) are being performed by simultaneously contacting said immobilized compound with known amounts of said first and said second target in solution, and wherein the relative amounts of first and second target binding to said immobilized compound are measured.
